(19) United States
(12) Patent Application Publication
(10) Pub. No.: US 2021/0386091 A1
(43) Pub. Date: Dec. 16, 2021

(54) FEED INGREDIENT COMPRISING BUTYRATE AND A SELECTED FERMENTATION PRODUCT

(71) Applicant: SUPERBREWED FOOD, INC., New Castle, DE (US)

(72) Inventors: Bryan P. TRACY, Wilmington, DE (US); Daniel Knox MITCHELL, Wilmington, DE (US); John Randall PHILLIPS, Middletown, DE (US); Alon KARPOL, Tel-Mond (IL); Aharon M. EYAL, Jerusalem (IL)

(21) Appl. No.: 17/410,482

(22) Filed: Aug. 24, 2021

Related U.S. Application Data
(63) Continuation-in-part of application No. PCT/IB2020/051607, filed on Feb. 25, 2020.
(60) Provisional application No. 62/810,408, filed on Feb. 26, 2019.

(51) Int. Cl. A23K 10/12; A23K 20/105; A61K 35/74; A61K 31/19; A61K 9/00

(52) U.S. Cl. CPC A23K 10/12; A23K 20/105; A61K 9/0056; A61K 31/19; A61K 35/74

(57) ABSTRACT

Provided is a composition comprising a) butyrate and b) a by-product of fermentation of a carbon source-containing feedstock with a Clostridia class bacterium which natively produces butyric acid. Further provided are a feed ingredient, an animal feed and an anti-icing product comprising the composition, methods of manufacturing and uses thereof.

### FIELD OF THE INVENTION

The present invention relates to animal feeds, and more specifically to a feed ingredient comprising a) butyrate and b) a by-product of fermentation of a carbon source-containing feedstock with a Clostridia class bacterium which natively produces butyric acid, and methods of use thereof.

### BACKGROUND

There is an ongoing need for improved feed ingredients that better achieve improvements, such as increased feed intake, improved feed conversion rate, faster daily weight gain, greater ileum surface area and consistent results between sexes when fed to an animal.

### SUMMARY OF THE INVENTION

According to an aspect of some embodiments of the present invention, provided is a composition comprising butyrate and a by-product of fermentation of a carbon source-containing feedstock with a Clostridia class bacterium which natively produces butyric acid.

According to a further aspect of some embodiments of the present invention, provided is a feed ingredient comprising the composition as disclosed herein.

According to a further aspect of some embodiments of the present invention, provided is an animal feed comprising selected nutrients, the feed ingredient as disclosed herein and optionally biomass.

According to a further aspect of some embodiments of the present invention, provided is an anti-icing product comprising the composition as disclosed herein.

According to a further aspect of some embodiments of the present invention, provided is a method for manufacturing the composition as disclosed herein, the method comprising:

(i) providing said carbon-source-comprising feedstock; and
(ii) fermenting said feedstock with said Clostridia class bacteria, whereby a fermentation broth comprising a biomass, butyrate and the by-product is formed.

According to a further aspect of some embodiments of the present invention, provided is a feed ingredient comprising the composition as disclosed herein.

According to a further aspect of some embodiments of the present invention, provided is a method for treating an animal comprising feeding said animal with the feed disclosed herein.

According to a further aspect of some embodiments of the present invention, provided is method for manufacturing a feed ingredient, comprising

(i) providing a carbon-source-comprising feedstock;
(ii) fermenting said feedstock with a Clostridia class bacterium which natively produces butyric acid, whereby a fermentation broth comprising biomass is formed.

### BRIEF DESCRIPTION OF THE FIGURES

Some embodiments of the invention are described herein with reference to the accompanying FIGURES. The description, together with the FIGURES, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the FIGURES:

FIG. 1 is a line graph showing metabolite and cell production and glucose consumed as a function of fermentation time for Clostridium tyrobutyricum batch fermentation with glucose as the feedstock

### DETAILED DESCRIPTION OF THE INVENTION

The present invention relates to a composition comprising a) butyrate and b) a by-product of fermentation of a carbon source-containing feedstock with a Clostridia class bacterium which natively produces butyric acid, and methods of use thereof.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “feed ingredient” refers to a component, part, constituent or any combination/mixture to an animal food.

As used herein, the term “feedstock” refers to a component or components added to a fermentation

As used herein, the term “animal feed” refers to a product intended for consumption by a non-human animal, such as livestock, poultry, companion animal, or the like.

As used herein, the term “anti-icing material” refers to a material which can remove ice from a surface and/or prevent or reduce formation of ice on a surface.

As used herein, the term “protected” with regard to butyrate refers to a butyrate molecule that is provided in a bound or reversibly reacted form in order to enable the molecule to pass through the upper gastrointestinal tract.

As used herein, the term “treating” includes ameliorating, mitigating, and reducing the instances of a disease or condition, or the symptoms of a disease or condition.

As used herein, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed. As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

According to an embodiment, provided is a composition comprising butyrate and a by-product of fermentation of a carbon source-containing feedstock with a Clostridia class bacterium which natively produces butyric acid.

According to an embodiment, the composition further comprises acetate.

According to an embodiment, said organism said organism is selected from the group consisting of Butyribacterium methylotrophicum, Clostridium tyrobutyricum and combinations thereof.

According to an embodiment, the composition further comprises butyrate. According to an embodiment, said butyrate is protected for the transport through the stomach for higher yield of delivery to the intestine. According to an embodiment, said butyrate is bound to a glyceride. According to an embodiment, said butyrate is encapsulated or coated. According to an embodiment, said butyrate is protected by being encapsulated by a protein and/or by being bound to a protein. According to an embodiment, said butyrate forms between 10% by weight and 90% by weight of the composition on a dry basis. According to an embodiment, said butyrate forms at least 15%, at least 20% or at least 30% of the composition. According to an embodiment, said butyrate forms less than 80% of the composition, less than 70% or less than 60%.

According to an embodiment, said by-product of fermentation forms between 0.01% by weight and 90% by weight of the composition on a dry basis. According to an embodiment, said by-product of fermentation forms at least 0.05%, at least 0.1%, at least 0.2%, at least 1%, at least 2% at least 5% of the composition. According to an embodiment, said by-product is water soluble. According to an embodiment the solubility of said by-product in water at 20 degrees Celsius is greater than 3% by weight, greater than 5% wt, greater than 7% wt or greater than 9% wt.

According to an embodiment, said by-product is selected from the group consisting of nitrogen compounds, nucleotides, vitamins, salts, complexes of transition metals, biomass and combinations thereof. According to an embodiment, said biomass comprises an organism selected from the bacteria class of Clostridia. According to an embodiment, said biomass comprises an organism selected from Butyribacterium methylotrophicum, Clostridium tyrobutyricum and combinations thereof.

According to an embodiment, the composition further comprises a butyrate counter ion selected from the group consisting of ammonium, calcium, magnesium and combinations thereof.

According to an embodiment, said carbon source is selected from the group consisting of starch, dextrose, maltodextrin, glucose, liquefied corn mash, fructose, xylose, glycerol, sucrose, hemicellulose, cellulose and combinations thereof. According to an embodiment, said composition comprises less than 20% water, less than 15%, less than 10% or less than 5%.

According to an aspect of some embodiments of the present invention, there is provided a feed ingredient comprising the composition as disclosed herein.

According to an aspect of some embodiments of the present invention, there is provided an animal feed comprising selected nutrients and the feed ingredient as disclosed herein. According to some such embodiments, the animal feed further comprises biomass.

According to an aspect of some embodiments of the present invention, there is provided an anti-icing product comprising the composition as disclosed herein. According to some embodiments, the anti-icing product further comprises a butyrate counter ion selected from the group consisting of calcium, magnesium and combinations thereof.

According to an aspect of some embodiments of the present invention, provided is a method for manufacturing the composition as disclosed herein, the method comprising (i) providing the carbon-source-comprising feedstock; (ii) fermenting said feedstock with said Clostridia bacteria, whereby a fermentation broth comprising a biomass, butyrate and a by-product is formed.

According to an embodiment, the method further comprises separating said biomass from said fermentation product to form a clarified fermentation broth, wherein said clarified fermentation broth comprises butyrate and said by-product. According to an embodiment, said separating comprises at least one of filtering and centrifuging.

According to an embodiment, the carbon source is selected from the group consisting of starch, dextrose, maltodextrin, glucose, liquefied corn mash, fructose, xylose, glycerol, sucrose, hemicellulose, cellulose and combinations thereof.

According to an embodiment, said fermenting comprises pH control by adding to said fermentation broth a base selected from the group consisting of ammonia, urea, a calcium base, a magnesium base and combinations thereof.

According to an embodiment, said fermenting comprises pH control by adding ammonia to said fermentation broth and wherein said clarified fermentation broth comprises ammonium butyrate, the method further comprising:
adding to said clarified fermentation broth at least one selected from the group consisting of a calcium base and a magnesium base, whereby ammonia vapors are formed, and
collecting said ammonia vapors.

According to an embodiment, said collected ammonia vapors are used for said pH control.

According to an embodiment, the method further comprises adding butyrate to said clarified fermentation broth.

According to an embodiment, the method further comprises protecting at least a fraction of the butyrate in said clarified fermentation broth or in products thereof.

According to an embodiment, the method further comprises at least partially removing water from said fermentation broth and/or from said clarified fermentation broth.

According to an aspect of some embodiments of the present invention, there is provide a method for treating an animal comprising feeding said animal with the feed as disclosed herein.

According to an aspect of some embodiments of the present invention, there is provided the feed as disclosed herein for use in treating an animal.

According to an embodiment, said feed comprises butyrate at a concentration between 10 ppm and 20,000 ppm.

According to an embodiment, at least 50% by weight of the butyrate in said feed results from said feed ingredient.

According to an embodiment, said animal is selected from the group consisting of avian, porcine, bovine, equine, ovine, piscine, caprine, canine, feline and lapine groups of animals.

According to an embodiment, the method provides an improved result compared with feeding same selected nutrients without said feed ingredient.

According to an embodiment, said improved result comprises at least one of increased feed intake, improved feed conversion rate, faster daily weight gain, greater ileum surface area, and consistent results between sexes.

According to an embodiment, provided is a method for manufacturing a feed ingredient, comprising (i) providing a carbon-source-comprising feedstock; (ii) fermenting said feedstock with a Clostridia class bacterium which natively produces butyric acid, whereby a fermentation broth comprising butyrate and biomass is formed.

According to an embodiment, the method further comprises separating said biomass from said clarified fermentation broth to form a clarified fermentation broth, wherein said clarified fermentation broth comprises butyrate. According to an embodiment, said separating comprises at least one of filtering and centrifugation.

According to an embodiment, said carbon source is selected from the group consisting of starch, dextrose, maltodextrin, glucose, liquefied corn mash, fructose, xylose, glycerol, sucrose, hemicellulose, cellulose and combinations thereof.

According to an embodiment, said fermenting is anaerobic. According to an embodiment, said fermenting is conducted at pH greater than 4.8, greater than 5, greater than 5.2, greater than 5.4 or greater than 5.6. According to an embodiment, said fermenting is conducted at a temperature in the range between 25 and 40 degrees Celsius. According to an embodiment, said fermenting is conducted for between 10 hours and 50 hours after inoculation. According to an embodiment, said fermenting is continuous. According to an embodiment, said fermenting is conducted in a fed-batch mode.

According to an embodiment, said fermenting comprises pH control by adding to said feedstock a base selected from the group consisting of ammonia, urea, a calcium base, a magnesium base and combinations thereof.

According to an embodiment, said fermenting comprises pH control by adding ammonia to said feedstock and wherein the clarified fermentation broth comprises ammonium butyrate, the method further comprising: adding to said clarified fermentation broth at least one selected from the group consisting of a calcium base and a magnesium base, whereby ammonia vapors are formed, and collecting said ammonia vapors. According to some such embodiments, said collected ammonia vapors are used for said pH control.

According to an embodiment, said method further comprises adding butyrate to said clarified fermentation broth. According to an embodiment, said method further comprises protecting at least a fraction of the butyrate in said clarified fermentation broth or in products thereof. According to an embodiment, said method further comprises at least partially removing water from said fermentation broth and/or from said clarified fermentation broth. According to an embodiment, said water removal comprises water evaporation.

According to an aspect of some embodiments of the present invention, there is provided a method for treating an animal comprising feeding said animal with the feed as disclosed herein.

According to an aspect of some embodiments of the present invention, there is provided the feed as disclosed herein for use in treating an animal.

According to an embodiment of the method, the nutrients of the feed comprise energy sources and protein sources. According to an embodiment, said feed comprises butyrate at a concentration between 10 ppm and 20,000 ppm. According to an embodiment, said feed comprises at least 50 ppm butyrate, at least 100 ppm, at least 300 ppm, at least 500 ppm butyrate, at least 700 ppm, or at least 1000 ppm. According to an embodiment, at least 50% by weight of the butyrate in said feed results from said feed ingredient, at least 60%, at least 70% or at least 80%.

According to an embodiment, said animal is selected from the group consisting of avian, porcine, bovine, equine, ovine, piscine, caprine, canine, feline and lapine groups of animals.

According to an embodiment, treating is of a condition selected from the group consisting of poor feed intake, poor feed conversion rate, poor daily weight gain, insufficient ileum surface area, and variation in results between sexes.

According to an embodiment, the method is characterized by providing an improved result compared with feeding the same selected nutrients without the feed ingredient.

According to an embodiment, said improved result comprises at least one, at least two, at least three, or at least four selected from the group consisting of increased feed intake, improved feed conversion rate, faster daily weight gain, greater ileum surface area, consistent results between sexes and combinations thereof.

According to an embodiment, said provides an improved result compared with feeding same selected nutrients without said feed ingredient. According to an embodiment, said improved result comprises increased feed intake. According to an embodiment, feed intake increases by at least 0.5%, at least 1%, at least 2%, at least 3%, or at least 4%. According to an embodiment, said improved result comprises improved feed conversion rate (FCR). According to an embodiment, FCR improves by at least 0.1%, at least 0.2%, at least 0.3%, at least 0.4%, at least 0.5%, at least 1%, at least 2.5%, at least 3%, at least 4%, at least 5%, at least 6%, at least 7%, at least 8%, at least 9%, or at least 10%.

According to an embodiment, said improved result comprises faster daily weight gain. According to an embodiment, daily weight gain increases by at least 0.1%, at least 0.2%, at least 0.3%, or at least 0.4%.

### EXAMPLES

### Example 1: Glucose Fermentation

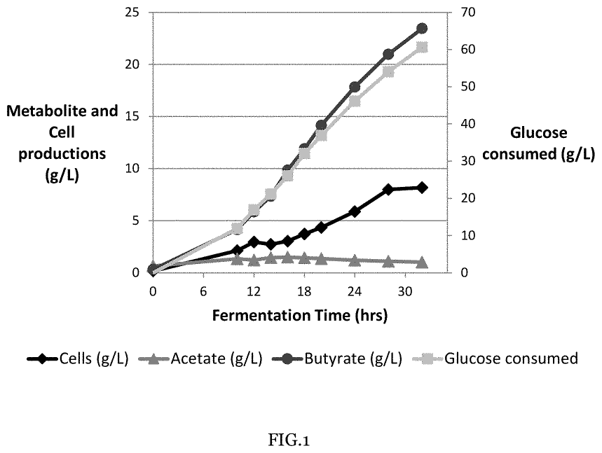

A glucose-based fermentation media, containing 60-160 g/L of glucose, was fermented in a batch fermentation with Clostridium tyrobutyricum. Additional media components include those listed in Tables 1, 2, and 3. Temperature was maintained at 35° C. and pH was controlled at minimum of 5.7 with 8M NH₄OH. Results of glucose consumption, cell production and metabolite production during 30 hours of fermentation are shown in FIG. 1. Butyrate concentration and cell concentration reached about 60 g/L and 20 g/L, respectively.

TABLE 1
Bulk media components.

| Component | Concentration (g/L) |
|---|---|
| KH2PO4 | 3 |
| K2HPO4*3H2O | 0.1975 |
| NaCl | 0.6 |
| MgSO4*7H2O | 0.24 |
| MnSO4*H2O | 0.01 |
| FeSO4*7H2O | 0.03 |
| (NH4)2SO4 | 1.5 |
| CaCl2*2H2O | 0.16 |
| Antifoam | 0.1 |

TABLE 2
Trace elements added to media.

| Component | Concentration (mg/L) |
|---|---|
| MnSO4*H2O | 20 |
| Fe(SO4)2(NH4)2*6H2O | 16 |
| CoCl2*6H2O | 4 |
| ZnSO4*7H2O | 0.004 |
| CuCl2*2H2O | 0.4 |
| NiCl2*6H2O | 0.4 |
| Na2MoO4*2H2O | 0.4 |
| Na2SeO4 | 0.4 |
| Na2WO4 | 0.4 |

TABLE 3
Vitamins added to media.

| Component | Concentration (mg/L) |
|---|---|
| Biotin | 0.04 |
| Folic Acid | 0.04 |
| Pyridoxine-HCl | 0.2 |
| Thiamine-HCl | 0.1 |
| Riboflavin | 0.1 |
| Nicotinic acid | 0.1 |
| Calcium-D-(+)-pantothenate | 0.1 |
| Vitamin B12 | 0.002 |
| p-Aminobenzoic acid | 0.1 |
| Thioctic acid | 0.1 |

### Example 2: Manufacture of the Feed Ingredient

Fermentation was conducted similarly to Example 1. Biomass was filtered out. The clarified fermentation broth was concentrated up by water evaporation to reach total solids concentration of 52.6% by weight to form the feed ingredient. Its density was 1.076 Kg/L. The formed feed ingredient was analyzed. The results are summarized in Table 4.

TABLE 4
Example composition of butyrate feed ingredient

| Component | Value | Units |
|---|---|---|
| Total solids | 52.6 | wt % |
| Density | 1.076 | kg/L |
| Ammonium Butyrate | 361.0 | g/kg |
| Ammonium Acetate | 112.0 | g/kg |
| Crude Protein (on AA basis) | 35.5 | g/kg |
| Crude Fat | 4.0 | g/kg |
| Ash | 5.5 | g/kg |
| Calcium | 0.1 | g/kg |
| Phosphorous | 0.4 | g/kg |
| Magnesium | 0.4 | g/kg |
| Potassium | 2.0 | g/kg |
| Sodium | 0.2 | g/kg |
| Iron | 6.0 | ppm |
| Zinc | 5.0 | ppm |
| Copper | <1 | ppm |
| Manganese | 20 | ppm |
| Molybdenum | 0.7 | ppm |
| Sulfur | 200 | ppm |

Amino acids (hydrolyzed values)

| | | |
|---|---|---|
| Lysine | 1.13 | g/kg |
| Methionine | 1.59 | g/kg |
| Threonine | 0.52 | g/kg |
| Aspartic acid/asparagine | 2.75 | g/kg |
| Glutamic acid/glutamine | 5.85 | g/kg |
| Serine | 0.66 | g/kg |
| Histidine | 0.30 | g/kg |
| Glycine | 1.17 | g/kg |
| Arginine | 0.68 | g/kg |
| Alanine | 13.47 | g/kg |
| Tyrosine | 0.45 | g/kg |
| Valine | 4.04 | g/kg |
| Phenylalanine | 0.74 | g/kg |
| Isoleucine | 0.65 | g/kg |
| Leucine | 0.93 | g/kg |
| Proline | 0.55 | g/kg |

### Example 3: Corn-Mash Fermentation

A fermentation similar to Example 1 was conducted except that instead of glucose, corn mash was used as the feedstock. Corn mash was diluted and filtered to a concentration of 60-160 g/L assumed glucose, and an amylase enzyme was added during the fermentation. Only trace elements (Table 2) and vitamins (Table 3) were added to the fermentation. Other operating conditions were the same and a similar production profile was achieved with a similar product as Example 2.

1. A composition comprising a) butyrate and b) a by-product of fermentation of a carbon source-containing feedstock with a Clostridia class bacterium which natively produces butyric acid.

2. An animal feed ingredient or an anti-icing product comprising the composition of claim 1.

3. The animal feed ingredient or anti-icing product of claim 2, further comprising acetate.

4. The animal feed ingredient or anti-icing product of claim 2, wherein said organism is selected from the group consisting of Butyribacterium methylotrophicum, Clostridium tyrobutyricum and combinations thereof.

5. The animal feed ingredient or anti-icing product of claim 2, wherein said by-product is selected from the group consisting of nitrogen compounds, nucleotides, vitamins, salts, complexes of transition metals, biomass and combinations thereof.

6. The animal feed ingredient or anti-icing product of claim 2, wherein said butyrate is protected.

7. The animal feed ingredient or anti-icing product of claim 2, comprising between 10% by weight and 90% by weight on a dry basis of said butyrate.

8. The animal feed ingredient or anti-icing product of claim 2, comprising between 0.01% by weight and 90% by weight on a dry basis of said by-product.

9. The animal feed ingredient or anti-icing product of claim 2, comprising less than 20% water.

10. An animal feed comprising selected nutrients, the feed ingredient of claim 2 and optionally biomass.

11. A method for manufacturing the animal feed ingredient or anti-icing product of claim 2, comprising
(i) providing said carbon-source-comprising feedstock; and
(ii) fermenting said feedstock with said Clostridia bacteria, whereby a fermentation broth comprising a biomass, butyrate and said by-product is formed; and
(iii) separating said biomass from said fermentation broth to form a clarified fermentation broth, wherein said clarified fermentation broth comprises butyrate and said by-product.

12. The method of claim 11, wherein said fermenting comprises pH control by adding ammonia to said fermentation broth and wherein said clarified fermentation broth comprises ammonium butyrate, the method further comprising:
adding to said clarified fermentation broth at least one selected from the group consisting of a calcium base and a magnesium base, whereby ammonia vapors are formed;
collecting said ammonia vapors; and
reusing said collected ammonia vapors for said pH control.

13. The method of claim 11, further comprising protecting at least a fraction of the butyrate in said clarified fermentation broth or in products thereof.

14. The method of claim 11, further comprising at least partially removing water from said fermentation broth and/or from said clarified fermentation broth.

15. A method for treating an animal comprising feeding said animal with the feed of claim 10.

16. The method of claim 15, wherein said feed comprises butyrate at a concentration between 10 ppm and 20,000 ppm.

17. The method of claim 15, wherein at least 50% by weight of the butyrate in said feed results from said feed ingredient.

18. The method of claim 15, wherein said animal is selected from the group consisting of avian, porcine, bovine, equine, ovine, piscine, caprine, canine, feline and lapine groups of animals.

19. A method for treating a condition associated with at least one selected from the group consisting of poor feed intake, poor feed conversion rate, poor daily weight gain, insufficient ileum surface area, and variation in results between sexes, comprising feeding said animal with the feed of claim 10.
